(19) United States
(12) Patent Application Publication
(10) Pub. No.: US 2011/0039834 A1
(43) Pub. Date: Feb. 17, 2011

(54) NOVEL PHARMACEUTICAL COMPOSITIONS FOR OPTIMIZING SUBSTITUTION TREATMENTS AND EXTENDING THE PHARMACOPOEIA TO GLOBAL TREATMENT OF ADDICTIONS

(75) Inventor: Mario Sanchez, Paris (FR)

Correspondence Address: BANNER & WITCOFF, LTD., 1100 13th STREET, N.W., SUITE 1200, WASHINGTON, DC, 20005-4051, US

(73) Assignee: TRIMARAN LIMITED, Hertfordshire (GB)

(21) Appl. No.: 12/278,622

(22) PCT Filed: Feb. 19, 2007
(86) PCT No.: PCT/IB2007/000390

§ 371 Date: Sep. 27, 2009

(30) Foreign Application Priority Data
Feb. 17, 2006 (FR) PCT/FR06/00372

(51) Int. Cl. A61K 31/40; A61K 31/519; A61K 31/165; A61K 31/451; A61K 31/5517; A61K 31/485; A61K 31/439; A61P 25/30

(52) U.S. Cl. 514/220; 514/428; 514/259.41; 514/619; 514/327; 514/282; 514/289; 514/304

(57) ABSTRACT

The present invention relates to the field of life needs and more particularly to the therapeutic field.

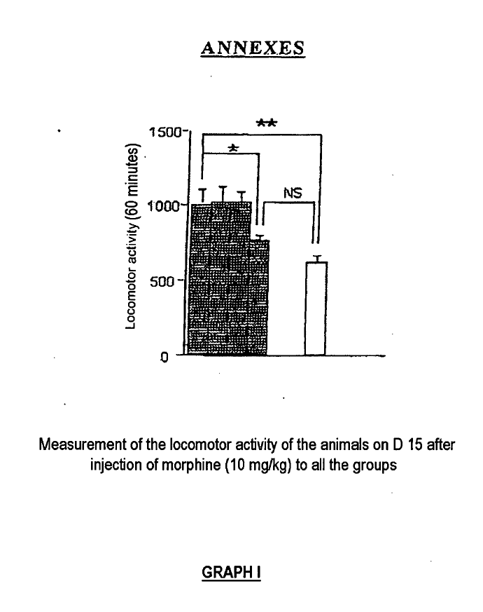

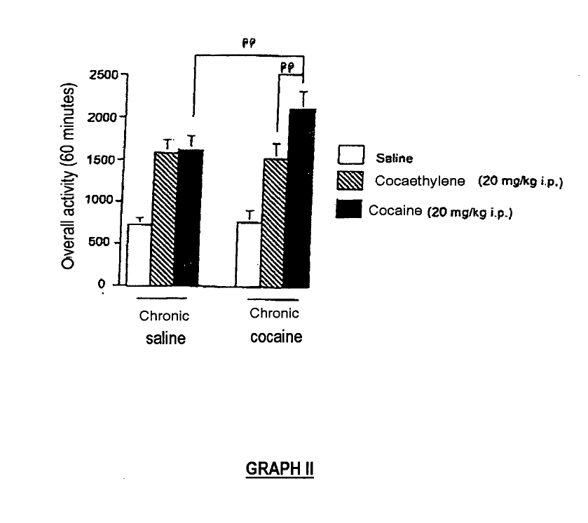

The invention relates more particularly to pharmaceutical compositions for helping the takers of addictive drugs to return to abstinence, in the form of a combination of two medicaments constituted of a partial or complete ligand of dopaminergic receptors—in particular of the D1, D2 and D3 receptors, and having direct prodopaminergic activity, and of an indirect prodopaminergic product, in the form of a pharmaceutical composition for oral, parenteral or transdermal administration.

The invention also relates to a method for combating the various forms of addiction to licit or illicit drugs.

The invention relates to the field of life needs and more particularly to the therapeutic field.

The invention relates more particularly to pharmaceutical compositions for helping, in a powerful manner, the takers of addictive drugs to return to abstinence and thus to lead them towards regaining normal social and/or professional activity.

Addiction (or dependency) may be defined as a behavioral disorder, characterized by a compulsive search for the product that causes this dependency, despite the harmful consequences on the health, family, professional life, etc. of which the dependent person is fully aware.

This loss of behavioral control appears as a result of repetitive consumption, but, in the case of heroin and opiates, the transition from abuse of these substances to addiction may be very brief. This depends on a certain number of genetic and environmental parameters intrinsic to each individual.

This dependency is due to the excessive and repeated stimulation of the opioid receptors, in particular of mu type (Mattes et al. Nature, 1996, 383, 819-823), more particularly in the cerebral structures forming the limbic system (ventral tegmental area, nucleus accumbens, amygdala, prefrontal cortex, etc.). Changes gradually follow in the functioning of the neurons, which maintain this dependency and, most especially, induce a very powerful and very long-lasting remnance of the effects of the substance.

These effects are characterized by sedation, euphoria or a reduction in the inner tensions of the consumer. Furthermore, there is an “orgasmic” pleasure effect, known as a “rush”, which follows, for example, the injection of heroin. The effect of this substance and of opioids, or other highly addictive drugs such as cocaine, leads, on coming down, to overexcitment of the neuronal control systems that produce an inverse effect, i.e.: anxiety, dysphoria, etc. This inverse effect appears in particular during the stopping of consumption of the drug: this is the “withdrawal syndrome”, which is very painful and, in most cases, short-lived, giving rise to repetitive relapses.

One of the ways of reducing these very painful states that “hook” the dependent person on his drug is to seek to stabilize the patient by avoiding the ecstatic “rush” states, and by treating the cause of the major behavioral disorders that led to the addiction.

The most spectacular successes have been obtained by substituting heroin, or other addictive opioids, with substances that are also capable of stimulating the opioid receptors, but less powerfully, and for different reasons. For some of them, it is a problem of pharmacokinetics that leads to a long, slow impregnation of the brain with this opioid substance. As a result, the receptors are never powerfully stimulated, as they are with heroin, but they are stimulated sufficiently for the patient not to suffer from a lack and from an uncontrollable urge to obtain the “substance” (craving). This is the case for methadone, a full agonist, which has been used as a heroin substitution treatment since 1964 in the USA, and was approved by the FDA in 1973. Another substance that is increasingly used is buprenorphine, which is a partial agonist of the mu opioid receptors with a long duration of action. As a result, buprenorphine is incapable, even at high doses, of producing the “rush” described previously.

These substitution treatments give noteworthy results, but suffer from a major defect. They only lead to an often relative reduction in the addictive behavior. Replacement therapies have enabled the cognizance and consideration by the medical world of the pharmacological treatment of heroin dependency. Their contribution is incontrovertible, not only in France but throughout the world.

The prior art (US 2005/014 786, US 2005/171 110, US 2005/080 087, WO 2005/016 286) already describes a specific action on the cannabinoid CB₁-CB₂ receptors in an antagonist form, whereas the present patent application describes a direct and indirect simultaneous agonist modulation of the D₁, D₂ and/or D₃ dopaminergic receptors.

This concept is sufficiently different from that of the preceding documents since, according to the invention, dopaminergic agonist opioid substances, as may be methadone or buprenorphine, act on the same receptors and in the same way as those on which stupefying drugs act. Unlike replacement treatments and contrary to the opinion put forward in the documents mentioned previously, according to which the products act in an opposite manner to the stupefying effect of cannabinoids, the compositions according to the invention act rather in the same way as naloxone. The present invention should find a therapeutic application starting from the modulation of the receptors concerned and a particular potential and kinetic stimulation, rather than from an antagonist action.

Specifically, the invention described in the reference US 2005/01476A1 is not precisely a treatment of addiction, but should be considered as a broader psychiatric treatment ranging from schizophrenia to anxiety, within which there might be a place for the treatment of addiction, since the use of cannabis is a major problem in this field. However, the lack of specificity permits the description of any psychotropic substance as a potential treatment of addiction, but the specificity is largely unproven after the agonist action (blocking agent) of the molecule and of the drug responsible for the abuse. In other words, the prior-art references deal mainly with the symptomatic signs of the disease, which is confirmed by the authors of the prior references (US 2005/171 110): cannabinoid receptor modulators can reduce or improve the abuse of a substance or addiction disorders. This is why a combination of cannabinoid receptor modulators with medicaments used for treating addictive disorders may lead to a dose reduction or improve the efficacy of therapeutics for common addiction-related disorders.

The present invention is thus a determined combination of treatments that will optionally be used for various psychiatric applications, but that are above all intended for treating addiction. This does not mean that the various compounds of the invention might be added to other already-used medicaments, but that the specific combination of these two molecules will work better than one or the other, taken individually on a population of patients that cannot be defined beforehand as psychotic or by another known diagnosis.

This invention has the result of modifying the current therapeutic maintenance schemes, in contrast with the others for which the doses increase until sedation appears. The principle of the present treatment consists in installing a second molecule that essentially affords the maintenance treatments the lowest possible but necessary dose.

The prior-art references describe a treatment that might be added to the regular existing treatments. They do not describe it as a treatment that might guarantee obtaining, via the combination of two particular components, a better therapeutic effect than that obtained with one or the other of the components administered individually.

This is why, in contrast with the prior references according to which antidopaminergic medicaments such as haloperidol or olanzapine may be combined with a conventional antipsychotic medicament, the present invention details the fact that a twofold action on a given dopaminergic system can treat drug addicts who correspond to a diagnosis of psychosis.

The present invention draws its efficacy from direct and indirect simultaneous actions, the effect of which is, precisely, an action on the dopaminergic neurons.

Reference WO 2004/100 992 (Pfizer) describes a novel component for improving antipsychotic treatments of the “new generation” type, for instance a treatment with a conventional antipsychotic agent to which the authors of the said reference add either benzodiazepines or GABAergic modulators.

The said reference does not describe either the treatment of addicts or the treatment of addiction, but an improvement in the treatment of psychosis.

The therapeutic principle according to which an agonist and a dopaminergic antagonist are added together and not an agonist+another agonist is described herein, but neither buprenorphine nor methadone is featured in the supposedly exhaustive list of molecules described by the preceding authors.

Reference WO 2005/181 071 describes an innovation for obtaining an improvement in the treatment of depression in which depressions that are resistant to standard treatments are thought to result from a novel hypothesis concerning the transmission of monoamines. The said reference does not describe either the treatment of addicts or the treatment of addiction, but describes an improvement in the treatment of psychosis. The inventor counts either on the dopaminergic antagonist+SSRI (selective serotonin reuptake inhibitor) combination or on mood regulators+SSRI. The therapeutic principle thus combines an agonist and an antagonist, although there is absolutely no description of such a principle.

More precisely, neither of the two active principles is a direct agonist of the dopaminergic system. Specifically, the authors use an indirect agonist among one of the two (SSRI) which is a very slow or even delayed dopaminergic agonist. This contributes towards definitively denaturing and differentiating this system from that which is the subject of the present patent application. In particular, neither buprenorphine nor methadone is listed as dopaminergic agonist in the supposedly exhaustive list of dopaminergic agonist molecules given in the reference US 2005/181 071.

In summary, the present invention does not relate to an effect on the cannabinoid receptors (CB1 and CB2). In addition, the present invention does not relate to two opposite actions (agonists or antagonists), but essentially above all to two simultaneous agonist actions. A stable dopaminergic action brought about by these simultaneous actions should result therefrom. To obtain such a result, a combination of direct and indirect activators is used. A “direct action” means an action by which an administered molecule directly activates a reaction on the dopaminergic neurons, which may be of inhibition or stimulation. An “indirect action” means that neuromodulators are activated, which will secondarily (or consequently) stimulate the dopaminergic neurons.

One subject of the present invention is thus a well-determined combination of treatments that may optionally be declined for psychiatric applications, but which relates most particularly to addiction phenomena. This does not mean that the molecules in question might be added to others that are already used, but that the claimed specific combination of two molecules will work more efficiently than one or the other taken individually on a population of patients that cannot be defined beforehand as psychotic, or for other known diagnoses.

The object of the present invention is to modify the current therapeutics of replacement treatments (OST maintenance), which the others do not do: a usual principle consists in increasing the doses until sedation appears. According to the present technique, it is preferable to install a second molecule so as to set the replacement treatments at their lowest possible but, however, necessary dosage level.

These therapeutic means and the development of neurosciences have amplified knowledge in the broader field of addictions.

Despite this, persistent disorders that occur in the course of a replacement treatment, in the case of hundreds of thousands of patients, indicate the limit of their efficacy. Although it is relatively easy to become abstinent from a drug, resisting it is more difficult.

The development of research in these fields has not provided, to date, any precise indication regarding treatments that may be transposed to other products capable of producing an addiction. It is known, however, that all these products act on three fundamental systems (opioid, GABAergic and dopaminergic systems) and that they all ultimately produce an increase in dopaminergic transmission.

In the present patent application, the invention has been based on the principle that it is important to use simultaneously two direct and indirect prodopaminergic molecules in dependent patients. This specific regulation will allow a gradual return to a neuronal situation closer to stability, aiding the re-establishment of the systems that had been deregulated by the repeated taking of compounds during the addiction period. This will be valid for opiate replacement treatments, but also for supplements.

The invention that is the subject of the present patent application lies in the fact that, contrary to all expectations, the treatment of individuals who are dependent on heroin and on opioids and also, to a lesser extent, on psychostimulants (for example cocaine), by the combination of a dopaminergic receptor ligand with a prodopaminergic action (referred to hereinbelow as a direct prodopaminergic agent), in particular of the D2 and/or D3 type, and of a second compound that may be termed prodopaminergic, which does not act directly on the dopaminergic receptors but indirectly modifies the release of dopamine (opioid—including methadone, buprenorphine, LAM (laevo-alpha-acetylmethadol) or all the other substances claimed as having this property of acting on the opioid receptors, etc.) (referred to hereinbelow as an indirect prodopaminergic agent), leads to a rapid improvement in the state of internal psychic tension that leads to the compulsive search for the addictive substance.

Thus, the combination, during the administration of the two substances (a direct prodopaminergic and an indirect prodopaminergic) is capable of producing an anti-addiction effect, at least during the first weeks of treatment.

Without this finding being fully explained, it is the fruit of experimental clinical studies.

The improvement in the physical state of the dependent individuals is such that it very rapidly allows the establishment of a search for the underlying causes of the compulsive behavior, characteristic of the addiction.

One subject of the invention is thus, specifically, a pharmaceutical composition containing a combination of two medicaments, preferably in kit form, intended to be administered, simultaneously or successively, for facilitating withdrawal, which consists of a combination of two agonists: one a direct prodopaminergic agent in particular on the D1, D2 and D3 receptors, and the other an indirect prodopaminergic agent (capable of modulating the release of dopamine), including opioid replacement products, in the form of a pharmaceutical composition for oral, parenteral or transdermal administration.

The dopaminergic agonist is preferably an agonist of the D1, D2 or D2/D3 type.

Among the dopaminergic agonists, the molecules most widely used are:
amisulpride (at a prodopaminergic dose),
risperidone,
olanzapine.

Other dopamine antagonist substances, such as sulpiride, metoclopramide, or even olanzapine or haloperidol, may also be used.

The indirect prodopaminergic agonist may be defined as a substance capable of binding or not binding to or in the opioid receptors, which only weakly manifests euphoric activity and/or which manifests only a limited addiction effect. Mention may be made in this regard of methadone, buprenorphine, the product known as LAM, nalorphine, naltrexate, levallorphan and, in general, any substance described as having such a property. It may also be cocaethylene.

The invention thus lies in the administration of such a combination either simultaneously in the form of a single defined pharmaceutical composition, or in the form of a kit containing each of said active principles in a separate form, which may thus be administered at variable dosages, or at different rhythms or in a different order, or in different forms.

The combination of the two active principles may thus be administered in two identical pharmaceutical forms, for instance plain tablets, gel capsules, sugar-coated tablets or drops, or in different forms, for instance gel capsules of different colors or drops of solutes of different composition.

The concentrations of active principles may also vary, passing from a high dose to a lower dose, as a function of the therapeutic needs, the pursuit of the treatment and the occurrence of side effects.

It is already known practice to use amisulpride or salts thereof, and especially S-(−)-amisulpride for the treatment of affective or cognitive symptoms of schizophrenia, for the treatment of autism or for the treatment of neuroleptic-induced tardive dyskensia

(PCT/EP99/05325). Patent PCT/EP99/05325 also mentions that S-(−)-amisulpride may be used to counter “drug addiction” without any other details.

Amisulpride is one of the many representatives of the benzamide series described in patent U.S. Pat. No. 4,401,822 as an anti-apomorphine substance. The synthesis of amisulpride in racemic or enantiomerically pure [S(−)] form is described in patent application PCT/EP99/05325, along with that of salts thereof.

Amisulpride is described in pharmacology as displacing [³H]-raclopride from the limbic D2 receptors. As a result of its central action, amisulpride may be considered as an antipsychotic medicament in the case of individuals suffering from schizophrenia, most especially by manifesting fewer side effects than the known antipsychotic neuroleptic products, such as extrapyramidal syndrome, etc.

Amisulpride is thus a known medicament, but one that has been used hitherto in other neuropsychiatric indications.

The effect of the medicaments that are the subject of the present combination appears rapidly and, already in preclinical studies, a positive effect is noted taking into account the impregnation effect.

The doses administered in the context of the pharmaceutical compositions according to the invention will be variable as a function of the desired effect, the length of existence of the dependency on the addictive drugs and the intensity of the action against the desired addiction.

The doses of direct prodopaminergic substances may range from 1 mg to 600 mg per unit intake. The doses of the indirect prodopaminergic compound will range from 0.2 mg to 2000 mg per unit intake, and preferentially from 0.2 mg to 300 mg per unit intake.

In one preferred embodiment of the invention, the combination will be formed of tablets of direct prodopaminergic compound, containing from 25 mg to 500 mg of active principle, and of tablets of indirect prodopaminergic substance, at a dose of 0.2 mg to 500 mg per unit intake. The direct prodopaminergic compound is preferentially amisulpride. The indirect prodopaminergic substance is preferentially chosen from methadone, buprenorphine, the product known as LAM, nalorphine, naltrexate, levallorphan and cocaethylene.

Another embodiment that is particularly useful will be the presentation in the form of a kit containing, for example, two bottles of a solid or liquid preparation, one of the bottles containing a solution of direct prodopaminergic substance, the other bottle containing a solution or a suspension of indirect prodopaminergic substance, for instance an aqueous methadone suspension or syrup.

In another embodiment of the combination according to the invention, combined forms may be produced, especially dry forms containing the two active principles and thus achieving a simultaneous administration. Two-coat plain tablets or two-core sugar-coated tablets containing, in one of the parts of the pharmaceutical form, the direct prodopaminergic substance, and, in the other part, the indirect prodopaminergic substance, may thus be envisioned. Scored tablets are also an easy administration form.

Injectable forms may also be prepared. They allow the simultaneous administration of the two active principles of the combination. They are justified in particular for preparing deposit forms with sustained action. Transdermal forms may also be envisioned with a sustained effect.

It is also possible to prepare fixed combinations containing determined doses of each of the active principles either in free form, or in physically combined form, or in chemically combined form, for instance a double salt with a polycarboxylic acid, or with an acidic resin. These combinations will have to be fixed during clinical trials, but they will be less easy to use if wide distribution of the products and the production of favorable results are to be targeted.

The usual dosage regimen generally consists in using low doses of prodopaminergic medicament, and then in gradually increasing the doses to obtain a “plateau” effect.

In the case of amisulpride, the daily dosage will range from 50 to 500 mg, with a single intake of 50 to 400 mg.

In the case of risperidone, the dosage will range from 1 to 4 mg per day.

The administration of indirect prodopaminergic products, and especially of methadone, will range from 5 to 120 mg per dosage intake. The doses of buprenorphine, morphine sulfate or nalorphine will be of a proportionally equivalent order of magnitude.

The order of administration of the two components of the combination according to the invention is not a determining factor and may be modified according to the therapeutic needs. It would appear preferable to ensure the administration firstly of the indirect prodopaminergic substance, and then of the direct prodopaminergic product. It is possible, on the other hand, to administer the direct prodopaminergic product first, then followed by administration of the indirect prodopaminergic product. In any case, it is more convenient for the administration of the two active principles to be simultaneous.

A subject of the invention is also a pharmaceutical composition constituted of a combination of a direct prodopaminergic product or a salt thereof and of an indirect prodopaminergic product or a salt thereof, containing, for example, from 50 to 500 mg of amisulpride and from 0.2 to 30 mg of indirect prodopaminergic product in a pharmaceutically acceptable non-toxic inert excipient or vehicle. The dosage is modulated by increasing at first, and then, when the threshold effect is reached, the dosage of one and/or the other of the prodopaminergic products is reduced. The indirect prodopaminergic product is chosen from naltrexone, nalorphine and buprenorphine, preferentially buprenorphine.

Another subject of the invention lies in the production of a kit containing a first pharmaceutically suitable dosage of direct dopaminergic substance, for instance amisulpride, in base form or in salt form, in racemic form or in enantiomeric form, at a dose of from 100 to 400 mg, and a second pharmaceutically suitable dosage of methadone containing from 5 to 200 mg of methadone per single intake.

Another subject of the invention lies in the production of pharmaceutical compositions constituted of a combination of risperidone and of an indirect prodopaminergic agent chosen from nalorphine, methadone, buprenorphine and nallorphan, such that the dose of risperidone is from 1 to 4 mg per unit intake.

The invention also relates to an anti-addiction medicament consisting of the combination of sulpiride in racemic or optically active form, and in free form or salified with a mineral or organic acid, and of buprenorphine.

According to another embodiment of the invention, the pharmaceutical compositions may also contain a neuroleptic agent, for instance a phenothiazine.

The combination according to the invention is intended to be administered at a rate of once or twice a day, exceptionally three times a day, to ensure constant impregnation of the individual with medicament.

The pharmacological and clinical trials, the details of which are given in the appendix, show the efficacy of the combination according to the invention.

The invention also relates to a method for combating the different forms of addiction to licit or illicit drugs, which consists in administering to individuals displaying phenomena of addiction to the licit or illicit drugs a sufficient and effective amount of a combination of a direct dopaminergic agonist and of an indirect prodopaminergic agonist simultaneously, in a single or separate pharmaceutical form, or alternatively in batch form, by first administering the indirect prodopaminergic compound, in a given pharmaceutical form, followed by the direct dopaminergic agonist in another pharmaceutical form, for example in kit form.

The method described above is most particularly suitable for combating addiction to opiate drugs, for instance heroin. It also finds a use in combating the use or abuse of active principles that lead to addiction, for instance amphetamine and derivatives thereof, alcohol, cocaine and NMDA.

### EXPERIMENTAL SECTION

Specific objectives of the study:

1. to demonstrate that the use of the combination of amisulpride at low dose (direct prodopaminergic action) and of buprenorphine (indirect prodopaminergic action) makes it possible to reduce the behavioral sensitization to morphine following a chronic administration;

2. to demonstrate the advantage of using an indirect prodopaminergic compound in a formulation allowing slow release.

1. Opiates and the opioid system

1.1 The opioid receptors

Activation of the opioid receptors produces a wide variety of physiological and pharmacological responses. Specifically, the opioid system is involved especially in modulating stress, pain, mood, the cardiovascular function, and the taking of food (Vaccarino et al., 2000).

The use of radiolabeled ligands with high specific activity has allowed the discovery, in the central nervous system of mammals, of stereospecific, saturable and high-affinity receptors. These specific membrane binding sites for exogenous opiates were demonstrated by three teams (Simon et al., 1973; Terenius, 1973; Pert and Snyder, 1973). More recently, the receptors have been cloned and are defined as being of three types: δ, μ and κ (Kieffer et al., 1992; Chen et al., 1993; Yasuda et al., 1993). Depending on their sequence, it clearly appears that the opioid receptors belong to the major family of seven-domain transmembrane receptors binding the heterotrimeric G proteins (Dohlman et al., 1987). These receptors have 60% sequence homology in man, the most conserved sequences being the transmembrane domains and the intracellular loops. Furthermore, they are differently distributed in the central nervous system. The μ opioid receptors are widely present throughout the central nervous system, with very high concentrations in certain regions such as the basal ganglions, the limbic structures, the thalamic nuclei and regions important for nociception. The delta and kappa receptors have a more reduced distribution, and are most especially present in the ventral and dorsal striatum for the former, and in the dorsal striatum and the preoptic area for the latter (Mansour et al., 1988).

The signal transduction cascades associated with the opioid receptors have been widely studied in various tissues, cell types or neuronal preparations. It has been shown that these three receptors are coupled to the Gi/Go proteins which modulate numerous effectors. Specifically, the opioid receptors inhibit adenylate cyclase activity (Sharma et al., 1977), thus leading to a reduction in the level of intracellular cAMP, reduce the calcium conductance (Hescheler et al., 1987; Surprenant et al., 1990), stimulate the potassium channels (North et al., 1987) and increase the level of intracellular calcium (Jin et al., 1992). More recently, it has been shown that these receptors are capable of generating mitogenic signals by activating the MAP-kinase pathways (Fukada et al., 1996).

1.2 The endogenous opioid peptides

The endogenous ligands of the opioid receptors are the endomorphines (Hughes et al., 1975). These are neuropeptides released into the synaptic space, from large vesicles with a dense core, as a consequence of stimulation of neurons where they coexist with other neurotransmitters. Endomorphines are derived from distinct precursors and are heterogeneously present in the various populations of neurons of the central nervous system. Proopiomelanocortin (or POMC) gives rise to β-endorphin and to related peptides, proenkephalin A is the source of the enkephalins (Met- and Leu-enkephalin) and of similar peptides, and prodynorphin gives rise to the neo-endorphins and to dynorphin (Akil et al., 1998).

1.3 Enkephalin degradation enzymes and synthetic inhibitors of these enzymes

Enkephalins have a very short lifetime after their release (less than a minute). This brevity is not due, as for most of the standard neuromediators, to a reuptake system, but to their enzymatic degradation. Met-enkephalin (Try-Gly-Gly-Phe-Met) and Leu-enkephalin (Tyr-Gly-Gly-Phe-Leu) are rapidly hydrolyzed by cleavage of the Gly-Phe bond with a peptidase that was initially known as enkephalinase, which has since been demonstrated to be identical to neutral endopeptidase (NEP), and at the Tyr-Gly bond with aminopeptidase N (APN) (Rogues, 1986). These two enzymes belong to the same group of zinc metallopeptidases.

Many inhibitors of these enzymes have been synthesized in order to increase the lifetime of enkephalins, and thus to prolong their effects (Rogues, 1993). However, in order to fully protect the endogenous opioid peptides from enzymatic degradation, it is necessary to inhibit not only NEP but also APN (Bourgoin et al., 1986).

Several series of mixed enkephalin inhibitors have been developed (Rogues, 1986), including RB101, which is a molecule capable of crossing the blood-brain barrier (Fourni-Zaluski et al., 1992), but which is also endowed with low oral bioavailability.

Enkephalin catabolism inhibitors increase the extracellular concentration of enkephalins without affecting their release (Daugé et al., 1996; Bourgoin et al., 1986; Waksman et al., 1985). The advantage of these molecules is that, even at very high doses, they never induce pharmacological responses that are as powerful as that of morphine (Ruiz-Gayo et al., 1992; Abbadie et al., 1994), and are thus free of the standard side effects of opiates (constipation, dryness of the mouth, itching, irregular periods and, more seriously, gastrointestinal disorders and respiratory depression).

1.4 Opiates

The exogenous opioid receptor ligand that has been known for the longest time and that is used in medicine is morphine, an alkaloid derived from Indian poppy.

Other substances have the same pharmacological characteristics as morphine. Heroin (diacetylmorphine, diamorphine), which is metabolized to morphine, was introduced into medicine in 1898 in the treatment of tuberculosis. Nowadays, this substance is highly favored by drug addicts, due to its rapid penetration into the brain where it generates an orgasmic response, the “high”.

Other opiate agonists are nowadays used in substitution treatments: this is the case for methadone and buprenorphine. Methadone is a synthetic opiate and, like morphine, is a preferential agonist of the μ receptors.

Other synthetic opioids such as DAMGO and DPDPE are conventionally used as selective ligands of the μ and δ receptors, respectively, in experimental pharmacology (Handa et al., 1981; Mosberg et al., 1983).

Another class of exogenous opioid receptor ligands exists: the opioid antagonists. Mention may be made, inter alia, of naloxone, which is used therapeutically in the treatment of acute opiate intoxication. This molecule binds with the same affinity to the two μ and δ receptors. Another known antagonist is naltrindole, which binds with very strong affinity to the δ receptors (Fang et al., 1991). It is widely used in experimental pharmacology.

2. Addiction

2.1 Introduction: dependency or addiction

According to the WHO definition, dependency/addiction is a syndrome in which the consumption of a product becomes a requirement greater than that of other behaviors that were previously of maximum importance. Dependency becomes established with repetition of the taking of drugs and is characterized by a compelling need for the drug, which leads to its compulsive search. Dependency has two facets: physical and psychic.

The physical component obliges the drug addict to consume the drug at the threat of experiencing pain specific to the withdrawal syndrome (which, apart from exceptional cases, is not mortal, despite the strength of the pain experienced). It may disappear after a few days. The psychic component is the drug addict's desire to recommence, and is associated with a strong stimulation of the brain by the reinforcement/reward system and is the cause of many relapses in drug addiction. It may last for several years.

2.2 Opiate dependency and tolerance

Tolerance is the process of adaptation of a body to a substance, which is reflected by the gradual attenuation of the effects of said substance, and results in the need to increase the dose in order to obtain the same effects. In animals, tolerance results in a decrease in the behavioral effects induced by the drug following its repeated administration.

Chronic activation of the opioid system by exogenous ligands such as morphine leads to the establishment of a dependency characterized by the compulsive search for the drug. In animals, especially in rats, many experimental models have made it possible to demonstrate the behavioral effects of opiates. Techniques, such as self-administration or the conditioned preference of place, have demonstrated the reinforcing effects of heroin and morphine (McBride et al., 1999), which effects appear to be mainly mediated by the μ opioid receptors (Matthes et al., 1996).

2.3 Withdrawal

The abrupt interruption of consumption of drugs is manifested, in drug addicts, by physical and psychic symptoms. Withdrawal from opiates is manifested, inter alia, by hypertension and abdominal cramps, but also by anhedonism and dysphoria.

In animals, withdrawal from opiates may be brought about by the administration of an opioid antagonist, naloxone. Several behavioral changes are then observed in morphine-dependent rats: increase in grooming, mastication, blinking of the eyes, but also diarrhea or weight loss.

2.4 Theory of addiction (Robinson and Berridge)

Several theories have been postulated regarding addiction. One of them puts forward the hypothesis of a disassociation between the system subtending pleasure and that responsible for desire. Thus, the first leads to hedonic pleasure following a reward and is thought to be subtended by the opioid system. The second intervenes in the motivation and search for rewards (desire), and is thought to involve the mesolimbic dopaminergic neurons. The increase in compulsive behavior is thought to be due to sensitization of the latter neurons (Robinson-Berridge theory; see Robinson and Berridge, 2001).

The dopaminergic system is under the influence of many transmitters, inhibitors or activators. It has also been shown that many catecholaminergic, serotoninergic, glutamatergic, GABAergic, cholinergic and peptidergic systems undergo important changes in opiate dependency (Nieto et al., 2002, Ammon-Treiber et al., 2005).

Moreover, the endogenous opioid system plays an important role in addictive behavior. Thus, numerous studies have shown that genetically modified mice, which no longer express the gene encoding the opioid receptor of mu type, no longer develop dependencies, not only on opiates, but also on alcohol, cannabinoids and cocaine (Becker et al., 2002; Matthes et al., 1996). Moreover, it has been observed in mice no longer expressing the gene encoding the D2 dopaminergic receptor, which are mice that are incapable of developing an appetence for morphine (Maldonado et al., 1997), that they express a very high level of pre-proenkephalin, a precursor of enkephalins (endogenous opioid peptides) (Baik et al., 1995).

3. Dopaminergic system and amisulpride

3.1 The dopaminergic system

Dopamine acts on two classes of receptors: “D1-like” and “D2-like”. The D1-like receptors (D1 and D5) are coupled via Gs to adenylate cyclase and allow the production of cAMP, which triggers numerous metabolic responses dependent on protein kinase A. The D2-like receptors (D2, D3 and D4) are coupled to Gi/o and inhibit the synthesis of cAMP, which in particular facilitates opening of the hyperpolarizing K⁺ channels.

The dopaminergic neurons are organized in cellular groups, they are highly branched and innervate several structures of the brain. The two main dopaminergic groups located at the junction of the mesencephalon and of the diencephalon are the nigro-striatal system (designated by A8 and A9) and the mesocorticolimbic system (group A10).

The neurons A8 and A9 arise from the substantia nigra (ventrolateral part of the mesencephalon) and project onto the striatum. They play an essential role in regulating motor functions. Degeneration of these nigro-striatal neurons is responsible for Parkinson's disease (German et al., 1989).

The cellular bodies of the dopaminergic A10 (DA-A10) neurons are located in the ventral tegmental area (VTA) (Oades et al., 1987). They project onto all the structures of the limbic system: the nucleus accumbens, the olfactive tubercles, the cerebellar tonsil, the septum, the hippocampus and the frontal cortex. They directly or indirectly exchange information from the whole body, but most particularly with the cerebellar tonsil, the role of which in emotional perception is known, and with the hippocampus, which memorizes the sensations arising from the activation of this dopaminergic pathway. By simplifying to the extreme, it may be stated that they have an orchestrating role in the cerebral concert, in particular as regards mood, the hedonic value of stimuli (pleasure), wakefulness, attention, cognitive activity and memory. The productive manifestations of schizophrenia, manic episodes, outbursts of delirium and hyperkinesia in children appear to be associated with the hyperactivity of these neurons. On the other hand, the deficient manifestations of schizophrenia (anhedonia, withdrawal) and certain depressive states would correspond to their hypoactivity. Any increase in the activity of the A10 dopaminergic neurons, in particular in the region of the nucleus accumbens, is associated with sensations of pleasure.

It is possible to specifically destroy the A10 dopaminergic neurons via the intracerebral injection of a neurotoxin, 6-hydroxydopamine. Injection of this product into the region of the cellular bodies (in the ventral tegmental area), or into the regions into which they project (nucleus accumbens), irreversibly destroys these neurons. It has been shown that such a lesion of the A10 dopamine neurons produces a state of intense anhedonia. Moreover, the psychostimulating effects of cocaine, amphetamine and nicotine are no longer present and the place preference produced by these drugs is abolished (disappearance of the appetitive effects) in rodents. Finally, animals in which the dopamine neurons of the A10 area have been destroyed no longer self-administer these drugs (disappearance of the reinforcing effects). More specifically, it has been shown that it is the dopaminergic endings of the nucleus accumbens where dopamine is released that are involved in these appetitive and reinforcing effects (Fibiger et al., 1987; Zito et al., 1985; Shimura et al., 2002).

Contrary to what has just been seen, specific lesion of the noradrenalin or serotonin neurons does not attenuate the addictive power of these substances (Fletcher et al., 1999).

The dopamine neurons are mainly assembled in two mesencephalic nuclei. One is the ventral tegmentum or ventral tegmental area (VTA, or mesencephalic area A10), whose axonal projections innervate the cortex (especially in its anterior part), the limbic system (especially the septum and the amygdala) and basal nuclei (putamen and nucleus accumbens). The majority of these fibers pass through the median telencephalic fascicle (MTF) and are involved in the processing of cognitive-affective information.

In point of fact, this neuronal cabling belongs to the reinforcement/reward system, which produces a very strong cerebral stimulation in order to evoke pleasure (hedonic action) during behaviors essential to the survival of the species or of the individual. It is this motivation circuit that is bypassed by drugs. Thus, by producing pleasure, drugs motivate the individual towards compulsive behavior where drug use replaces the survival behaviors.

The other dopaminergic nucleus is the substantia nigra (locus niger or mesencephalic area A9) that emits axons toward the striatum (caudate nucleus and putamen) and that participates in controlling locomotion. Drugs that modify the level of release of dopamine in the striatum disrupt motor functions.

3.2 Dopamine-dependent mechanisms

The administration of morphine stimulates the activity of the dopaminergic neurons in the substantia nigra and in the VTA, which leads to an increase in dopamine release into the caudate nucleus-putamen and into the nucleus accumbens (Matthews and German, 1984; Spangel et al., 1990; Di Chiara and North, 1992).

It is commonly accepted that this increase is due to an indirect action of opioids. Specifically, activation of the g receptors present at the surface of the GABAergic interneurons located in the reticular substantia nigra and the VTA are believed to lead to removal of the inhibition exerted by these interneurons on the dopaminergic neurons (Johnson and North, 1992; Bontempi and Sharp, 1997).

3.3 Amisulpride, pro- or anti-antidopaminergic activity as a function of the dosages used

Amisulpride is a molecule chemically related to benzamides. At low doses, amisulpride has an antagonist effect on the D2 and D3 presynaptic receptors (net effect: facilitation) of the frontal cortex. In contrast, amisulpride used at high doses inhibits the post-synaptic D2 and D3 receptors (net effect: blockage) on the limbic system. Furthermore, it is free of extrapyramidal effects, since it has only low activity on the striatum (Perrault et al., 1996). All these factors make this molecule an atypical antipsychotic, which is nowadays used in the treatment of the positive and negative symptoms of schizophrenia.

### MATERIALS AND METHODS

1. Animals and treatments

The animals used in this study are male mice of OF1 strain weighing about 20 g at the start of the experiments (Charles River, France). They live in an environment whose daily lighting cycle (07:30h; 17:30h) is constant throughout the year, and the temperature is maintained at about 22° C. The mice have free access to water and food, and the experiments are performed in accordance with the international rules on ethics in animal experimentation.

2. Methods

2.1 Measurement of the locomotor activity

The mice are placed individually in a sound-insulated plastic cage (255 cm×205 cm) and are exposed to a light intensity of 5 lux. The animals' movements are captured by photoelectric cells for 60 minutes and recorded by computer. The experiment starts immediately after injection of the product. In this study, the term “locomotor activity” takes into account only the horizontal movements of the animals.

2.2 Behavioral sensitization:

Several theories have been postulated regarding addiction. One of them puts forward the hypothesis of a disassociation between the system subtending pleasure and that responsible for desire. Thus, the first leads to pleasure following a reward and is thought to be subtended by the opioid system. The second intervenes in the motivation and search for rewards (desire), and is thought to involve the mesolimbic dopaminergic neurons. The increase in compulsive behavior is thought to be due to sensitization of the latter neurons (Robinson-Berridge theory). The locomotor activity is influenced by a wide diversity of factors. However, a certain number of resemblances exists between the stimulating effects of drugs on locomotion and on motivation, suggesting that the locomotor activation observed following an administration of drug has a motivational origin.

3. Statistical analysis

A one-factor (treatment) analysis of variance (ANOVA) is used for all the behavioral tests performed, followed by a Student-Newman-Keuls test if p<0.05 in the ANOVA. In all the cases, the significance is accepted once p<0.05.

### RESULTS

1. Determination of the doses of amisulpride used

A molecule endowed with dopaminergic antagonist activity reduces locomotor activity. It is this property that is exploited in order to determine the dose after which amisulpride has dopaminergic antagonist activity in mice (i.e. an effect on the D2 and D3 post-synaptic receptors, and not on the D2 and D3 auto-receptors). The doses tested are: 0.5 mg/kg, 2 mg/kg, 10 mg/kg, 20 mg/kg and 50 mg/kg.

The decrease in locomotor activity is significant at and above 10 mg/kg. On the other hand, at low dose (0.5 mg/kg), locomotor hyperactivity is observed. This dose resulting in prodopaminergic responses is thus chosen for the rest of the experiments.

2. Amisulpride and buprenorphine combination: can the behavioral sensitization be reduced following a pretreatment with morphine

The experimental protocol used may be described in the following manner:

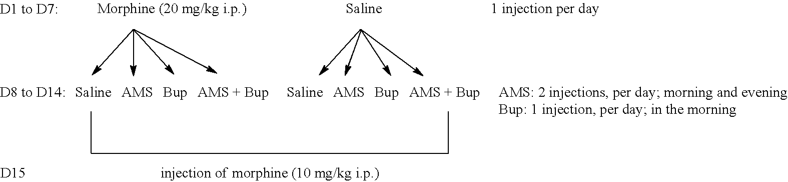

Dose of amisulpride (AMS): 0.5 mg/kg Dose of buprenorphine (Bup): 0.1 mg/kg

The results obtained show (see graph I):
1. The animals chronically treated with morphine from D1 to D7 show behavioral sensitization on D15 relative to the animals chronically treated with saline.
2. The installation of a treatment between D8 and D14 with amisulpride or buprenorphine administered alone does not make it possible to reduce this behavioral sensitization.
3. The installation of a treatment between D8 and

D14 with amisulpride+buprenorphine makes it possible to significantly reduce (p<0.05) the behavioral sensitization. It is interesting to note that there is no longer a significant difference with the control group.

3. Demonstration of the advantage of using a prodopaminergic compound in a formulation allowing slow release:

The repeated administration of cocaine in physiological saline results in a behavioral sensitization that appears very rapidly. After a weaning period of several days, followed by a new injection of cocaine, the result indicates that the animals still express this sensitization. This is the reason which drove the interest for forms with more broadly spread bioavailability of cocaine, such as cocaethylene. This property may be intensified by placing it in a suitable galenical form.

Thus, cocaethylene was placed in an oily solution, which is described to allow slow release of the product.

The protocol used may be illustrated in the following manner:

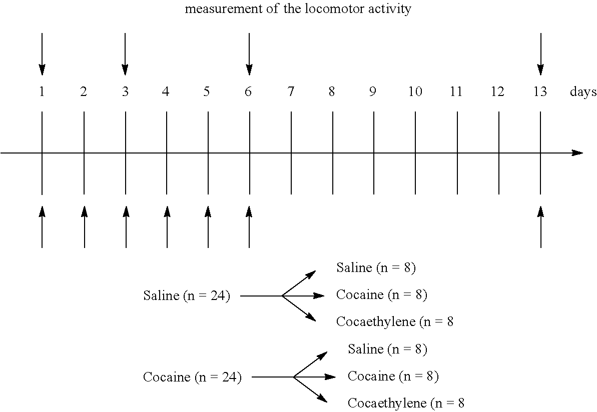

The animals are treated for 6 days with cocaine (20 mg/kg i.p.). The locomotor activity of the animals is measured for 1 hour immediately after the i.p. injection on D1, D3 and D6. Next, the animals are weaned for 6 days, before repeating an injection on D13 of saline, cocaine (20 mg/kg i.p.) or cocaethylene (20 mg/kg i.p.) and remeasuring the locomotor activity of the animals for 1 hour.

It is interesting to note that no behavioral sensitization is observed after chronic treatment with cocaethylene in emulsion, whereas the literature describes it when it is in solution in physiological saline (Prinssen et al., 1996). This clearly confirms that, with suitable kinetics, the dopaminergic system can be efficiently stimulated without, however, sensitizing it, as is the case with the modes of administration allowing a very large peak effect.

Moreover, it was clearly shown that animals pretreated with cocaine, which express a behavioral sensitization when the product is reinjected, do not express this behavior at all when cocaethylene in emulsion is injected. This result thus appears to indicate that cocaethylene in this pharmaceutical formulation does not result in any behavior that may be likened to euphoria in man, while at the same time, however, activating the dopaminergic system, since an increase in locomotor activity (without sensitization) is observed after administration of cocaethylene (see graph II).

Graph I shows the variations in the locomotor activity of the animals on Day D15 after injection of morphine at a dose of 10 mg/kg. The animals received from D1 to D7 either morphine or physiological saline, from Day D8 to D14 either physiological saline, or buprenorphine, amisulpride, buprenorphine and amisulpride according to the scheme:

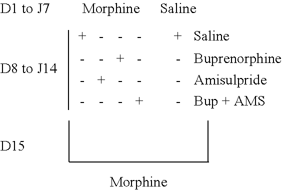

Graph II shows the overall level of locomotor activity measured over one hour. The animals were treated for 6 days with cocaine (20 mg/kg i.p.) or physiological saline. Next, the animals were weaned for 6 days before repeating an injection of cocaine (20 mg/kg i.p.), of cocaethylene (20 mg/kg i.p.) or of physiological saline. **p <0.01

### BIBLIOGRAPHY

Abbadie C, Honore P, Fournie-Zaluski M C, Rogues B P, Besson J M. Effects of opioids and non-opioids on c-Fos-like immunoreactivity induced in rat lumbar spinal cord neurons by noxious beat stimulation. Eur J Pharmacol. 1994 Jun. 13; 258(3):215-27.

Akil H, Owens C, Gutstein H, Taylor I, Curran E. Watson S. Endogenous opioids: overview and current issues. Drug Alcohol Depend. 1998 Jun.-Jul. 1; 51(1-2): 127-40.

Ammon-Treiber S. and V. Hollt, Morphine-induced changes of gene expression in the brain, Addict Biel 2005, 10: 81-9

Baik J. H., R. Picetti, A. Saiardi, G. Thiriet, A. Dierich, A. Depaulis, M. le Meur and E. Borrelli, Parkinsonian-like locomotor impairment in mice lacking dopamine D2 receptors, Nature 1995, 377: 424-428

Becker A., G. Grecksch, J. Kraus, H. H. Loh, IL Schroeder and V. Hollt, Rewarding effects of ethanol and cocaine in mu opioid receptor-deficient mice, Newlyn Schmiedebergs Arch Pharmacol 2002, 365: 296-302

Bontempi B, Sharp F R. Systemic morphine-induced Fos protein in the rat striatum and nucleus accumbens is regulated by mu opioid receptors in the substantia nigra and ventral tegmental area. Neurosci 1997 Nov. 1; 17(21):8596-612.

Bourgoin S, Le Bars D, Artaud F, Clot A M, Bouboutou R, Fournie-Zatuski M C, Rogues B P, Hamon M, Cesselin F. Effects of kelatorphan and other peptidase inhibitors on the in vitro and in vivo release of methionine-enkephalin-like material from the rat spinal cord. J Pharmacol Exp Ther. 1986 July; 238(1):360-6.

Chen Y, Mcatck A, Liu J, Hurley I A, Yu L. Molecular cloning and functional expression of a muoplaid receptor from rat brain. Mol Pharmacol 1993 Jul; 44(1):8-12.

Dauge V, Mauborgne A, Cesselin F, Fournie-Zaluski M C, Roque B P. The dual peptidase inhibitor RB101 induces :a long-lasting increase in the extracellular level of Met-enkephalin-like material in the nucleus accumbens of freely moving rats. J Neurochem. 1996 Sep; 67(3): 1301-8.

Di Chiara G, North R A. Neurobiology of opiate abuse. Trends Pharmacol Sci. 1992 May; 13(5):185-93.

Dohlman H G , Caron M G, Lefkowitz R J. A family of receptors coupled to guanine nucleotide regulatory proteins. Biochemistry. 1987 May 19; 26(10): 2657-64.

Fang L, Knapp R J, Horvath R, Matsunaga T O, Haaseth R C, Herby V I, Forma F, Yamamura H I. Characterization of [3H]naltrindole binding to delta opioid receptors in mouse brain and mouse vas deferens: evidence for delta opioid receptor heterogeneity. J Pharmacol Exp Ther. 1994 Feb; 268(2): 836-46.

Fibiger R C, F. G. LePiane, A. Jakubovic and A. G. Phillips, The role of dopamine in intracranial self-stimulation of the ventral tegmental area, Neurosci 1987, 7: 3888-96

Fletcher P. J., K M. Korth and J. W. Chambers, Depletion of brain serotonin following intra-raphe injections of 5,7-dihydroxytryptamine does not alter d-amphetainine self-administration across different schedule and access conditions, Psychopharmacology (Berl) 1999, 146: 185-93

Fournie-Zaluski M C, Cork P, Turcaud S, Lucas E, Noble F, Maldonado R, Rogues B P. “Mixed inhibitor-prodrug” as a new approach toward systemically active inhibitors of enkephalin. degrading enzymes. J Med Chem. 1992 Jun 26; 35(13):2473-81.

Fukuda K, Kato S, Morikawa H, Shoda T, Mori K. Functional coupling of the delta-, mu-, and kappa-opioid receptors to mitogen-activated protein kinase and arachidonate release in Chinese hamster ovary cells. I Neurochem. 1996 Sep; 67(3); 1309-16.

German D. C., K. Manaye, W. K. Smith, D. J. Woodward and C. B. Soper, Midbrain dopaminergic cell loss in Parkinson's disease: computer visualization, Ann Neurol 1989, 26: 507-14

Hands B K, Land A C, Lord J A, Morgan B A, Rance M J, Smith C F. Analogues of bets-LPH61-64 possessing selective agonist activity at mu-opiate receptors. Eur J Pharmacol 1981 Apr 9; 70(4):531-40.

Hescheler J, Rosenthal W, Trautwein W, Schultz G. The GTP-binding protein, Go, regulates neuronal calcium channels. Nature. 1987 Jan 29-Feb 4; 325(6103): 445-7.

Hughes I, Smith T W, Kosterlitz H W, Fothergill L A, Morgan B A, Morris H R. Identification of two related pentapeptides from the brain with potent opiate agonist activity. Nature. 1975 Dec 18; 258(5536): 577-80.

Jin W, Lce N M, Loh H H, Thayer S A. Upioids mobilize calcium from inositol 1,4,5-trisphosphate-sensitive stores in NG108-15 cells. J Neurosci. 1994 Apr, 14(4):1920-9.

Johnson S W. North R A Opioids excite dopamine neurons by hyperpolarization of local interneurons. J Neurosci. 1992 Feb; 12(2): 483-8.

Kieffer B L, Beton K, Gaveriaux-Ruff C, Hirth C G. The delta-opioid receptor: isolation of a cDNA by expression cloning and pharmacological characterization. Proc Natl Acad Sci USA. 1992 Dec 15; 89(24): 12048-52. Erratum in: Proc Natl Acad Sci USA 1994 Feb 1-91(3): 1193

Maldonado R., A. Saiardi, O. Valverde, T A. Samad, B. P. Rogues and E. Borrelli, Absence of opiate rewarding effects in mice lacking dopamine D2 receptors, Nature 1997, 388; 586-9

Mansour A, Khachaturian H, Lewis M E. Akil H, Watson S J. Anatomy of CNS opioid receptors. Trends Neurosci. 1988 Jul; 11(7): 308-14.

Matthes H W, Maldonado R, Simonin F, Valverde O, Slowe S, Kitchen I, Befort K. Dicrich A, Le Meur M, Dolle P, Tzavara E, Hanoune J, Rogues B P, Kieffer B L. Loss of morphine-induced analgesia, reward effect and withdrawal symptoms in mice tacking the mu-opioid-receptor gene. Nature. 1996 Oct 31; 383(6603): 819-23.

Matthews R T, German D C. Electrophysiological evidence for excitation of rat ventral tegmental area dopamine neurons by morphine. Neuroscience. 1984 Mar; 11(3): 617-2.5.

McBride W J, Murphy J M, Ikemoto S. Localization of brain reinforcement mechanisms: intracranial self-administration and intracranial place-conditioning studies. Bcbav Brain Res. 1999 Jun; 101(2): 129-52.

Mosberg H I, Hurst R, Hruby V J, Gee K, Yamamura H I, Galligan J J, Burks T F. Bis-penicillamine enkephalins possess highly improved specificity toward delta oploid receptors. Proc Natl Acad Sci USA. 1983 Oct; 80(19): 5871-4.

Nieto M. L, O. Caamano, F. Fernandez, M. Gomez, J. Balzarini and E. De Clercq, Synthesis, antiviral and cytostatic activities, of carbocyclic nucleosides incorporating a modified cyclopentane ring. IV. Adenosine and uridine analogues, Nucleosides Nucleotides Nucleic Acids 2002, 2): 243-55

North R A, Williams J T, Surprenant A, Christie M J. Mu and delta receptors belong to a family of receptors that arc coupled to potassium channels. Proc Natl Acad Sci USA. 1987 Aug: 84(15): 5487-91.

Oades R D. and G. M. Ventral tegmental (A10) system: neurobiology. 1. Anatomy and connectivity, Brain Res 1987, 434: 117-65

Perrault G, Depoorterc R, Morel E, Sanger D J, Scatton B. Psychopharmacological profile of Amisulpride: an antipsychotic drug with presynaptic D2/D3 dopamine receptor antagonist activity and limbic selectivity. J Pharmacol Exp Ther. 1997 Jan; 280(1): 73-82.

Pert C B, Snyder S H. Opiate receptor: demonstration in nervous tissue. Science. 1973 Mar. 9; 179(77): 1011-4.

Prinssen E. P., M. S. K Jeven and W. Koek, Repeated administration of cocaethylene induces context-dependent sensitization to its locomotor effects, Psychopharmacology (Berl) 1996, 124:300-5

Robinson T. E. and K C. Berridge, Incentive-sensitization and addiction,. Addiction 2001, 96: 103-14

Rogues B P, Fournie-Zaluski M C. Enkephalin degrading enzyme inhibitors: a physiological way to new analgesics and psychoactive agents. NIDA Res Monogr. 1986; 70:128-54.

Rogues B P. Zinc metallopeptidases: active site structure and design of selective and mixed inhibitors: new approaches in the search for analgesics and anti-hypertensives. Biochem Soc Trans. 1993 Aug; 21 (Pt 3)(3): 678-85.

Ruiz-Gayo M, Baamonde A, Turcaud S, Fournie-Zaluski M C, Rogues B P. In vivo occupation of mouse brain opioid receptors by endogenous enkephalins: blockade of enkephalin degrading enzymes by RB 101 Inhibits [3H] diprenorphine binding. Brain Res. 1992 Feb 7; 571(2):306-12.

Sharma S K, Klee W A, Nirenberg M. Opiate-dependent modulation of adenylate cyctasc. Proc Natl Acad Sci USA. 1977 Aug; 74(8): 3365-9.

Shimura T., Y. Kamada and T. Yamamoto, Ventral tegmental lesions reduce overconsumption of normally preferred taste fluid in rats, Behav Brain Res. 2002, 134: 123-30

Simon E J, Hiller J M. Edelman L Stereospecific binding of the potent narcotic analgesic (3H) Etorphine to rat-brain homogenate. Proc Natl Acad Sci USA. 1973 Jul; 70(7): 1947-9.

Spanagel R, Herz A, Shippenberg T S. The effects of opioid peptides on dopamine release in the nucleus accumbeur an in vivo microdialysis study. J Neurochem. 1990 Nov; 55(5): 1734-40.

Surprenant A, Shen K Z, North R A, Tatsumi H. Inhibition of calcium currents by noradrenaline, somatostatin and opioids in guinea-pig submucosal neurones. J Physiol. 1990 Dec; 431:585-608.

Terenius L. Characteristics of the “receptor” for narcotic analgesics in synaptic plasma membrane fraction from rat brain. Acta Pharmacol Toxicol (Copenh). 1973; 33(5)177-84.

Vaccarino A L, Kastin A J. Endogenous opiates: 2000. Peptides. 2001 Dec; 22(12): 2257-328.

Waksman G, Bouboutou R, Devin J, Bourgoin S, Cesselin F, Hamon M, Fournie-Zaluski MC, Rogues B. In vitro and in vivo effects of kelatorphan on enkephalin metabolism in rodent brain. Eur J Pharmacol. 1985 Nov 5; 117(2): 233-43.

Yasuda K, Raynor K, Kong H, Breder C D, Takeda J, Reisine T, Bell G I. Cloning and functional comparison of kappa and delta opioid receptors from mouse brain. Proc Natl Acad Sci USA. 1993 Jul 15; 90(14): 6736-40.

Zito K. A., G. Vickers and D. C. Roberts, Disruption of cocaine and heroin self-administration following kainic acid lesions of the nucleus accumbens, Pharmacol Biochem Behav 1985, 23: 1029-36

1. A pharmaceutical composition whose active principles are a combination of two medicaments intended to be used simultaneously or successively, constituted of a combination of a direct dopaminergic receptor agonist compound with prodopaminergic activity, which is a ligand of the D1, D2 and/or D3 receptors, and of a partial or complete indirect prodopaminergic product, characterized in that the direct dopaminergic receptor agonist compound is chosen especially from amisulpride, risperidone, sulpiride, metoclopramide, haloperidol and olanzapine.

2. The pharmaceutical composition as claimed in claim 1, in which the direct dopaminergic receptor agonist compound with prodopaminergic activity is a molecule also having a secondary serotonergic component, for instance olanzapine.

3. The pharmaceutical composition as claimed in claim 1, in which the direct dopaminergic receptor agonist compound with prodopaminergic activity is amisulpride in resolved form and especially S-(−)-amisulpride.

4. The pharmaceutical composition as claimed in claim 1, in which the indirect prodopaminergic product is a substance capable of binding to the opioid receptors or to systems capable of indirectly exciting the dopaminergic system, chosen from methadone, buprenorphine, the product known as LAM, nalorphine, naltrexate, cocaethylene and levallorphan.

5. The pharmaceutical composition as claimed in claim 1, which also contains a neuroleptic.

6. The pharmaceutical composition of claim 1, in which the combination of direct prodopaminergic and of indirect prodopaminergic product is in the form of a single defined pharmaceutical composition having a specific composition.

7. The pharmaceutical composition as claimed claim 1, in which the combination of a direct prodopaminergic and of indirect prodopaminergic product is in the form of a kit containing each of the active principles in a separate form.

8. The pharmaceutical composition as claimed in claim 1, in which the combination of the two active principles is in two identical pharmaceutical forms.

9. The pharmaceutical composition as claimed in claim 1, in which the combination of the two active principles is in two different pharmaceutical forms.

10. The pharmaceutical composition as claimed in claim 1, in which the doses of indirect prodopaminergic substance range from 0.2 to 2000 mg per single intake.

11. The pharmaceutical composition as claimed in claim 1, in which the doses of indirect prodopaminergic substance range from 0.2 mg to 300 mg.

12. The pharmaceutical composition as claimed in claim 1, in which the dose of racemic amisulpride or of amisulpride in the form of the S(−) isomer per single intake ranges from 50 mg to 400 mg.

13. The pharmaceutical composition as claimed in claim 1, characterized in that it is formed from tablets of amisulpride at a dose of from 25 mg to 500 mg and of tablets of indirect prodopaminergic substance chosen from nalorphine, methadone, buprenorphine, the product known as LAM, naltrexate, cocaethylene and levallorphan, at a dose of from 0.2 to 500 mg per single intake.

14. The pharmaceutical composition as claimed in claim 1, characterized in that it is in the form of a kit containing two bottles of a solid or liquid preparation of direct prodopaminergic substance, on the one hand, and of a liquid preparation of indirect prodopoaminergic substance, on the other hand.

15. The pharmaceutical composition as claimed in claim 1, consisting of a combination of amisulpride or a salt thereof, in racemic or enantiomerically pure form, and of methadone, characterized in that it contains from 100 to 400 mg of amisulpride and from 5 mg to 200 mg of methadone per single intake.

16. The pharmaceutical composition as claimed in claim 1, which is in the form of a kit containing a first pharmaceutically suitable dosage of amisulpride in base form or in salt form, in racemic form or in enantiomeric form, at a dose of from 50 mg to 500 mg, and a second pharmaceutically suitable dosage of buprenorphine containing from 0.2 to 30 mg per single intake.

17. The pharmaceutical composition as claimed in claim 1, consisting of a combination of risperidone and of an indirect prodopaminergic chosen from nalorphine, methadone, buprenorphine and nallorphan, characterized in that it contains from 1 to 4 mg of risperidone.

18. The pharmaceutical composition as claimed in claim 1, consisting of a combination of amisulpride and of buprenorphine, naltrexone or nalorphine, characterized in that it contains from 50 to 500 mg of amisulpride and from 0.2 to 30 mg of buprenorphine or naltrexone or nalorphine per single intake.

19-20. (canceled)

21. A method of preventing or different addition to licit or illicit drugs, comprising administering to an individual in need thereof an effective amount of the composition of claim 1.
